(19) United States
(12) Patent Application Publication
(10) Pub. No.: US 2009/0208977 A1
(43) Pub. Date: Aug. 20, 2009

(54) MATERIALS AND METHODS FOR SPERM SEX SELECTION

(75) Inventors: Keith HUDSON, Titirangi (NZ); Susan RAVELICH, Devenport (NZ)

Correspondence Address: SPECKMAN LAW GROUP PLLC, 1201 THIRD AVENUE, SUITE 330, SEATTLE, WA, 98101, US

(73) Assignee: ANDROGENIX LTD., Auckland City (NZ)

(21) Appl. No.: 12/178,531

(22) Filed: Jul. 23, 2008

Related U.S. Application Data
(60) Provisional application No. 60/951,363, filed on Jul. 23, 2007.
(60) Provisional application No. 61/029,835, filed on Feb. 19, 2008.

(51) Int. Cl. G01N 33/567; C12N 5/08

(52) U.S. Cl. 435/7.21; 435/366

(57) ABSTRACT

Materials and Methods for the separation of X- and Y-chromosome bearing sperm, for example in a semen sample, are provided. The methods involve contacting the semen sample with a binding agent, such as an antibody, that specifically binds to an antigen that is specific for an X- or Y-chromosome. Kits for use in the methods are also provided.

### REFERENCE TO RELATED APPLICATIONS

This application claims priority to U.S. Provisional Patent Application Nos. 61/029,835 filed Feb. 19, 2008, and 60/951,363 filed Jul. 23, 2007.

### FIELD OF THE INVENTION

This application relates to methods for identifying semen bearing the X or Y chromosome. More particularly, this application relates to sex-specific antigens and their use in such methods.

### BACKGROUND

The ability to identify and select male and female sperm has great value in the livestock industries, where there is an established market in artificial insemination of over US$ two billion per annum in the Organization for Economic Cooperation and Development (OECD). This is particularly true in the dairy industry where the majority of dairy farmers in key OECD markets impregnate their cows through artificial insemination. Sexed semen provides the opportunity to increase farmer productivity and income. For example, the availability of sexed semen would have significant impact in reducing and/or eliminating the minimal returns of male dairy calves as compared to female calves.

Genetic improvement, which has contributed significantly to increased milk yield per cow, is currently achieved by selecting the best sires and using artificial insemination (AI) to impregnate the herd. However, because the best cows can have either male or female progeny, the rate of genetic improvement is limited. The availability of sexed semen would allow selection of the best bulls and best cows from within a herd for herd replacement, thereby increasing the rate of genetic improvement. Utilizing sexed semen would also provide the opportunity to extend the average lactation length of high producing diary cows to 20-24 months, as replacements for these cows could be provided by less than two calves in a lifetime.

In the swine industry, semen sexing would remove the need for castration, improve feed efficiency and increase the lean meat content of the animals by reducing the number of males produced.

Currently the only available method to sort semen for sperm bearing the X or Y chromosome is to use a flow cytometer as described, for example, in U.S. Pat. Nos. 5,135,759, 5,985,216, 6,149,867 and 6,263,745. This approach exploits the small size difference in sperm size due to differences in DNA content to produce highly enriched populations of sperm with the X or Y chromosome (Johnson, Anim. Reprod. Sci. 60-61:93-107 (2000); Johnson et al., Biol. Reprod. 41:199-203 (1989)). However, this technique is limited by the use of the flow cytometer, and is too expensive and not easily scalable for use in routine sex selection in the livestock industry. Sexing semen by use of sperm surface molecules potentially provides a low cost, efficient and scaleable way to achieve this goal.

Previously used methods to detect surface differences on X & Y bearing sperm have been analytical, comprising a number of strategies, such as chromatography and immunological methods (Blecher et al., Theriogenology 52:1309-1321 (1999); Hendriksen et al., Mol. Reprod. Dev. 35:189-196 (1993); Howes et al., J. Reprod. Fertil. 110:195-204 (1997)). For example, U.S. Pat. No. 5,021,244 to Spaulding describes the use of flow cytometry followed by polyacrylamide gel electrophoresis (PAGE) to isolate sex-associated membrane proteins together with the use of such proteins to generate antibodies that can be employed to provide semen samples enriched in X or Y sperm. However, subsequent studies employing the methodology taught by Spaulding failed to identify any sex-specific spermatozoa, indicating that Spaulding's approach is unlikely to be successful (Howes et al., Jnl. Reproduction Fertility 110:195-204 (1997); Hendriksen et al., Mol. Reproduction Develop. 45:342-350 (1996)). US published patent application no. 2003/0162238 to Blecher et al. describes the isolation of a sex-chromosome-specific protein characterized as being X chromosome specific, associated with the cell membrane of bovine sperm cells and having a molecular weight of about 32 kDa.

The sensitivity of analytical techniques has recently improved with the introduction of two-dimensional-PAGE or multi-dimensional-chromatographic separation followed by mass spectrometry analysis (Domon and Aebersold, Science 312:212-217 (2006)). However, the analytical route still suffers from two major problems: first, that the most difficult group of proteins to analyze using this system are membrane components such as integral proteins, due to solubility issues; and second, that detection by mass spectrometry is limited in dynamic range. This limited dynamic range translates into a reduced sensitivity for detecting low abundance molecules if other high abundance species are present.

The methods described to date have been unsuccessful in discovering antigens specific for either X or Y bearing sperm, suggesting that either no differences exist and/or that the differences are small in nature and/or abundance. There thus remains a need in the art for materials and methods that may be effectively employed to identify and separate sperm bearing the X or Y chromosome.

### SUMMARY OF THE INVENTION

The present invention provides efficient, cost-effective and non-invasive methods for the identification and separation of X or Y-chromosome bearing sperm, together with compositions and kits for use in such methods. The disclosed methods have both high specificity (i.e. give few false positives) and high sensitivity (i.e. give few false negatives). The compositions disclosed herein comprise binding agents that specifically bind to antigens that are specific to either X- or Y-chromosome bearing sperm (referred to herein as X- or Y-chromosome specific antigens). The disclosed methods may be used in artificial insemination, for example, to increase the probability that offspring will be of the desired sex and/or to increase the probability that the offspring will carry a gene responsible for a desired trait.

In one aspect, methods for separating X- or Y-chromosome bearing sperm from semen are provided, together with sperm prepared by such methods. The disclosed methods comprise: (a) contacting the semen with at least one binding agent specific for an X- or Y-chromosome specific antigen for a period of time sufficient to form a conjugate between the binding agent and the X- or Y-chromosome bearing sperm; and (b) separating the conjugate from sperm which have not bound to the binding agent. The binding agent may be provided on a solid surface. In one embodiment, the binding agent is provided on the surface of a magnetic bead, such as a paramagnetic microsphere, and the binding agent-sperm conjugate is separated from non-bound sperm by applying an external magnetic field. In certain embodiments, the binding agents employed in such methods are specific for an antigen having an amino acid sequence selected from the group consisting of SEQ ID NO: 1-21, 43-89, 138, 140, 142, 144, 146, 148, 150, 152, 154, 156, 158, 160, 162, 164 and 183-201; sequences having at least 85%, 90%, 95%, 96%, 97%, 98% or 99% to a sequence of SEQ ID NO: 1-21, 43-89, 138, 140, 142, 144, 146, 148, 150, 152, 154, 156, 158, 160, 162, 164 and 183-201; and sequences encoded by a polynucleotide that hybridizes to a sequence of SEQ ID NO: 22-42, 90-136, 137, 139, 141, 143, 145, 147, 149, 151, 153, 155, 157, 159, 161, 163, 165-182 or 202 under stringent hybridization conditions.

In certain embodiments, the at least one binding agent employed in such methods is an antibody (such as a monoclonal antibody), or an antigen-binding fragment thereof, such as a Fab or scFv. Examples of binding agents that may be effectively employed in the disclosed methods include, but are not limited to, those provided in Table 1 below.

In another aspect, compositions comprising binding agents that are specific for an X- or Y-chromosome specific antigen are provided. In one embodiment, the binding agents are specific for an antigen having an amino acid sequence selected from the group consisting of SEQ ID NO: 1-21, 43-89, 138, 140, 142, 144, 146, 148, 150, 152, 154, 156, 158, 160, 162, 164 and 183-201, and variants thereof. Such binding agents may be labelled with a detection reagent and/or, as discussed above, attached to a magnetic bead in order to facilitate detection and/or separation of the X- or Y-chromosome bearing sperm in a biological sample, such as semen.

In a further aspect, kits for use in the disclosed methods are provided, such kits comprising a container holding at least one binding agent specific for an X- or Y-chromosome specific antigen disclosed herein. In certain embodiments, such kits comprise magnetic beads, such as paramagnetic microspheres, coated with, and/or attached to, one or more of the binding agents.

In yet another aspect, methods for identifying genes and/or proteins that are specific to X- or Y-chromosome bearing sperm are provided, such methods including a combination of bioinformatic and direct analytical steps as outlined in detail in the examples below. These methods may also be employed to identify surface differences between other closely related cells including, but not limited to, normal and cancer cells.

In a related aspect, methods for enriching a semen sample for either X- or Y-chromosome bearing sperm are provided, such methods comprising contacting a native semen sample with at least one binding agent disclosed herein, wherein binding of the X- or Y-chromosome bearing sperm to the binding agent is effective in reducing the mobility and/or activity of the sperm. In one embodiment, the disclosed binding agents may be conjugated to a cytotoxin using known methods, and used to destroy either X- or Y-chromosome bearing sperm. Such methods can be performed either in vitro in a semen sample, or in vivo by simultaneously or sequentially introducing a sperm sample and the binding agent into the vagina of a female animal. In certain embodiments, binding agents for use in such methods specifically bind to an antigen having an amino acid sequence selected from the group consisting of SEQ ID NO: 1-21, 43-89, 138, 140, 142, 144, 146, 148, 150, 152, 154, 156, 158, 160, 162, 164 and 183-201, sequences having at least 85%, 90%, 95%, 96%, 97%, 98% or 99% to a sequence of SEQ ID NO: 1-21, 43-89, 138, 140, 142, 144, 146, 148, 150, 152, 154, 156, 158, 160, 162, 164 and 183-201; and sequences encoded by polynucleotides that hybridize to a sequence of SEQ ID NO: 22-42, 90-136, 137, 139, 141, 143, 145, 147, 149, 151, 153, 155, 157, 159, 161, 163, 165-182 or 202 under stringent hybridization conditions.

These and additional features of the present invention and the manner of obtaining them will become apparent, and the invention will be best understood, by reference to the following more detailed description and the accompanying drawings.

### BRIEF DESCRIPTION OF THE FIGURES

FIG. 1 shows a comparison of RNA expression levels for known sperm proteins and orthologues of candidate X- or Y-chromosome specific genes disclosed herein.

FIG. 2 is a matrix of sperm treatment and binding assays employed in the present studies.

FIG. 3 is an outline of the SISCAPA technique used in the present studies.

FIG. 4 is an outline of the iTRAQ™ technique used in the present studies.

### DETAILED DESCRIPTION

The present disclosure provides antigens and variants thereof that are specific for either X- or Y-chromosome bearing sperm, together with binding agents that specifically bind to such antigens and/or variants thereof, and methods for the use of such binding agents in the detection and separation of X- and Y-chromosome bearing sperm. The amino acid sequences of disclosed bovine X- or Y-chromosome specific antigens are provided in SEQ ID NO: 1-21, 138, 140, 142, 144, 146, 148, 150, 152, 154, 156, 158, 160, 162 and 164, with the corresponding DNA sequences being provided in SEQ ID NO: 22-42, 137, 139, 141, 143, 145, 147, 149, 151, 153, 155, 157, 159, 161 and 163 respectively. The amino acid sequences of disclosed human X- or Y-chromosome specific antigens are provided in SEQ ID NO: 43-89, with the corresponding DNA sequences being provided in SEQ ID NO: 90-136, respectively. The amino acid sequences of equine X- or Y-chromosome specific antigens disclosed herein are provided in SEQ ID NO: 183-200, with the corresponding DNA sequences being provided in SEQ ID NO: 165-182, respectively.

A binding agent is herein defined as an agent that binds to an epitope of one of the disclosed X- or Y-chromosome specific antigens or a variant thereof, but does not bind detectably to unrelated polypeptides under similar conditions. Any agent that satisfies these requirements may be a binding agent. For example, a binding agent may be a polypeptide (such as a ligand), a ribosome (with or without a peptide component), an RNA molecule, or a small molecule. The ability of a binding agent to specifically bind to a polypeptide can be determined, for example, in a ELISA assay using techniques well known in the art, and/or using an assay described below in the Examples section. In preferred embodiments, a binding agent is an antibody, a functional antigen-binding fragment thereof, a small chain antibody variable domain fragment (scFv), a Fab fragment, a heavy chain variable domain thereof (V_{H}), or a light chain variable domain thereof (V_{L}). Binding agents that may be employed in the disclosed methods include, but are not limited to, those identified in Table 1.

TABLE 1

| Antigen | | | |
|---|---|---|---|
| SEQ | | | Catalog |
| ID NO: | Supplier | Antibody Name | number |
| 1 | SCBT* | PMCA3 (N-18) | SC-22074 |
| 1 | SCBT* | PMCA3 (C-15) | SC-22076 |
| 2 | SCBT* | BRS-3 (N-14) | SC-33404 |
| 2 | SCBT* | BRS-3 (K-19) | SC-33405 |
| 4, 138 | Made in-house | Anti-FAM11A | N/A |
| 5 | Made in-house | Anti-VSIG1 | N/A |
| 6, 140 | US Biological | CT 1 polyclonal | C7911-10 |
| | (Swampscott, MA) | antibody | |
| 7, 142 | SCBT* | ATP7A (C-20) | sc-30858 |
| 7, 142 | SCBT* | ATP7A (N-15) | sc-30856 |
| 7, 142 | SCBT* | ATP7A (H-180) | sc-32900 |
| 8 | SCBT* | XK (C-17) | sc-50198 |
| 8 | SCBT* | XK (W-13) | sc-50201 |
| 8 | SCBT* | XK (Y-16) | sc-50202 |
| 8 | IBGRL** | CD 238 antibody | 9440 |
| 8 | IBGRL** | CD 238 antibody | 9441 |
| 8 | R&D Systems, | Anti-human Kell | AF1914 |
| | Minneapolis, MN, USA | antibody | |
| 9 | SCBT* | NCAM-L1 (5G3) | SC-33686 |
| 9 | SCBT* | NCAM-L1 (I-18) | SC-31034 |
| 9 | SCBT* | NCAM-L1 (N-14) | SC-31032 |
| 9 | SCBT* | NCAM-L1 (UJ127.11) | SC-53386 |
| 9 | SCBT* | NCAM-L1 (H-200) | SC-15326 |
| 9 | SCBT* | NCAM-L1 (C-20) | SC-1508 |
| 10 | SCBT* | CXCR-3 (49801.111) | SC-57076 |
| 10 | SCBT* | CXCR-3 (C-20) | SC-6226 |
| 10 | SCBT* | CXCR-3 (H-95) | SC-13951 |
| 10 | SCBT* | CXCR-3 (CN-15) | SC-9900 |
| 12, 148 | Everest Biotech Ltd., | Anti-ATP6IP2/Renin | EB06118 |
| | Oxford, UK | receptor Antibody | |
| 13 | Everest Biotech Ltd., | Goat Anti-PGRMC1/ | EB07207 |
| | Oxford, UK | MPR Antibody | |
| 15 | SCBT* | CKR-3 (5E8) | SC-32777 |
| 15 | SCBT* | CKR-3 (H-52) | SC-7897 |
| 15 | R&D Systems, | Anti-human CCR3 | MAB155 |
| | Minneapolis, MN, USA | antibody | |
| 16, 152 | Medical & Biological | CX3CR1 | D070-3 |
| | Laboratories Co. Ltd., | | |
| | Woburn, MA, USA | | |
| 16, 152 | SCBT* | CX3CR1 (H-70) | SC-30030 |
| 16, 152 | SCBT* | CX3CR1 (K-13) | SC-31561 |
| 16, 152 | SCBT* | CX3CR1 (T-20) | SC-20432 |
| 21, 158 | Proteintech Group Inc., | FMR1NB antibody | 11069-2-AP |
| | Chicago, IL, USA | | |
| 45 | Made in-house | Anti-EFBN1 | N/A |
| | | extracellular domain | |

*Santa Cruz Biotechnology Inc., Santa Cruz, CA USA.
**International Blood Group Reference Laboratory, Bristol, UK.

In alternative embodiments, the binding agent is a protein. For example, the proteins CCL11, CC124 and CCL26 may be employed as binding agents for the antigen of SEQ ID NO: 15; CX3CL1 and fractaline may be used as binding agents for the antigen of SEQ ID NO: 16; CxCL9, CxCL1O and CxCL11 may be used as binding agents for the antigen of SEQ ID NO: 10; and rennin may be used as a binding agent for the antigen of SEQ ID NO: 12.

An “antigen-binding site”, or “antigen-binding fragment” of an antibody refers to the part of the antibody that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein, Eur. J. Immunol. 6:511-519, 1976, and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding the monoclonal antibodies disclosed herein may be isolated and sequenced using conventional procedures. Recombinant antibodies, antibody fragments, and fusions and polymers thereof, can be expressed in vitro or in prokaryotic cells (e.g. bacteria) or eukaryotic cells (e.g. yeast, insect or mammalian cells) and further purified as necessary using well known methods.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064). A method for construction of human combinatorial libraries useful for yielding functional Fab fragments has been described by Rauchenberger et al. (J. Biol. Chem. 278:38194-38205, 2003).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

A wide variety of expression systems are available in the art for the production of antibody fragments, including Fab fragments, scFv, V_{L} and V_{H}s. For example, expression systems of both prokaryotic and eukaryotic origin may be used for the large-scale production of antibody fragments and antibody fusion proteins. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins. Yeasts and filamentous fungi are accessible for genetic modifications and the protein of interest may be secreted into the culture medium. In addition, some of the products comply with the GRAS (Generally Regarded as Safe) status in that they do not harbor pyrogens, toxins, or viral inclusions.

Methylotrophic and other yeasts such as Candida boidinii, Hansenula polymorpha, Pichia methanolica, and Pichia pastoris are well known systems for the production of heterologous proteins. High levels of proteins, in milligram to gram quantities, can be obtained and scaling up to fermentation for industrial applications is possible.

The P. pastoris system is used in several industrial-scale production processes. For example, the use of Pichia for the expression of scFv fragments as well as recombinant antibodies and fragments thereof, has been described. Ridder et al., Biotechnology 13:255-260 (1995); Anadrade et al., J. Biochem. (Tokyo) 128:891-895 (2000); Pennell et al., Res. Immunol. 149:599-603 (1998). In shake-flask cultures, levels of 250 mg/L to over 1 g/L of scFv or V_{HH} can be achieved (Eldin et al., J. Immunol. Methods 201:67-75 (1997); Freyre et al., J. Biotechnol. 76:157-163 (2000)).

Similar expression systems for scFv have been described for Saccharomyces cerevisiae, Schizosaccharomyces pombe, Yarrowia lipolytica, and Kluyveromyces lactis. Horwitz et al., Proc. Natl. Acad. Sci. USA 85:8678-8682 (1988); Davis et al., Biotechnology 9:165-169 (1991); and Swennen et al., Microbiology 148:41-50 (2002). Filamentous fungi, such as Trichoderma and Aspergillus, have the capacity to secrete large amounts of proteins. This property may be exploited for the expression of scFv and V_{HH}s. Radzio et al., Process-biochem. 32:529-539 (1997); Punt et al., Trends Biotechnol. 20:200-206 (2002); Verdoes et al., Appl. Microbiol. Biotechnol. 43:195-205 (1995); Gouka et al., Appl. Microbiol. Biotechnol. 47:1-11 (1997); Ward et al., Biotechnology 8:435-440 (1990); Durand et al., Enzyme Microb. Technol. 6:341-346 (1988); Keranen et al., Curr. Opin. Biotechnol. 6:534-537 (1995); Nevalainen et al., J. Biotechnol. 37:193-200 (1994); Nyyssonen et al., Biotechnology 11:591-595 (1993); and Nyyssonen et al., International Patent Publication no. WO 92/01797.

In certain embodiments, the binding agents specifically bind to a variant of an X- or Y-chromosome specific antigen disclosed herein. As used herein, the term “variant” comprehends nucleotide or amino acid sequences different from the specifically identified sequences, wherein one or more nucleotides or amino acid residues is deleted, substituted, or added. Variants may be naturally occurring allelic variants, or non-naturally occurring variants. Variant sequences (polynucleotide or polypeptide) preferably exhibit at least 80%, 85%, 90%, 95%, 96%, 97%, 98% or 99% identity to a sequence disclosed herein. The percentage identity is determined by aligning the two sequences to be compared as described below, determining the number of identical residues in the aligned portion, dividing that number by the total number of residues in the inventive (queried) sequence, and multiplying the result by 100.

In addition to exhibiting the recited level of sequence identity, variants of the disclosed X- or Y-chromosome specific antigens are preferably themselves specific to either X- or Y-chromosome bearing sperm.

Variant sequences generally differ from the specifically identified sequence only by conservative substitutions, deletions or modifications. As used herein, a “conservative substitution” is one in which an amino acid is substituted for another amino acid that has similar properties, such that one skilled in the art of peptide chemistry would expect the secondary structure and hydropathic nature of the polypeptide to be substantially unchanged. In general, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Variants may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptide and polynucleotide sequences may be aligned, and percentages of identical nucleotides in a specified region may be determined against another polynucleotide, using computer algorithms that are publicly available. Two exemplary algorithms for aligning and identifying the identity of polynucleotide sequences are the BLASTN and FASTA algorithms. The alignment and identity of polypeptide sequences may be examined using the BLASTP and algorithm. BLASTX and FASTX algorithms compare nucleotide query sequences translated in all reading frames against polypeptide sequences. The FASTA and FASTX algorithms are described in Pearson and Lipman, Proc. Natl. Acad. Sci. USA 85:2444-2448, 1988; and in Pearson, Methods in Enzymol. 183:63-98, 1990. The FASTA software package is available from the University of Virginia, Charlottesville, Va. 22906-9025. The FASTA algorithm, set to the default parameters described in the documentation and distributed with the algorithm, may be used in the determination of polynucleotide variants. The readme files for FASTA and FASTX Version 2.0x that are distributed with the algorithms describe the use of the algorithms and describe the default parameters.

The BLASTN software is available on the NCBI anonymous FTP server and is available from the National Center for Biotechnology Information (NCBI), National Library of Medicine, Building 38A, Room 8N805, Bethesda, Md. 20894. The BLASTN algorithm Version 2.0.6 [Sep. 10, 1998] and Version 2.0.11 [Jan. 20, 2000] set to the default parameters described in the documentation and distributed with the algorithm, is preferred for use in the determination of variants according to the present invention. The use of the BLAST family of algorithms, including BLASTN, is described at NCBI's website and in the publication of Altschul, et al., “Gapped BLAST and PSI-BLAST: a new generation of protein database search programs,” Nucleic Acids Res. 25:3389-3402, 1997.

The “hits” to one or more database sequences by a queried sequence produced by BLASTN, BLASTP, FASTA, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The percentage identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the present invention; and then multiplying by 100 to determine the percentage identity.

In an alternative embodiment, variant polypeptides are encoded by polynucleotide sequences that hybridize to a disclosed polynucleotide under stringent conditions. Stringent hybridization conditions for determining complementarity include salt conditions of less than about 1 M, more usually less than about 500 mM, and preferably less than about 200 mM. Hybridization temperatures can be as low as 5° C., but are generally greater than about 22° C., more preferably greater than about 30° C., and most preferably greater than about 37° C. Longer DNA fragments may require higher hybridization temperatures for specific hybridization. Since the stringency of hybridization may be affected by other factors such as probe composition, presence of organic solvents and extent of base mismatching, the combination of parameters is more important than the absolute measure of any one alone. An example of “stringent conditions” is prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

All of the binding agents and X- or Y-chromosome specific antigens disclosed herein are isolated and purified, as those terms are commonly used in the art. Preferably, the binding agents and antigens are at least about 80% pure, more preferably at least about 90% pure, and most preferably at least about 99% pure.

The binding agents disclosed herein may be effectively employed in the separation of X- and Y-chromosome bearing sperm and can therefore be used to enrich a semen sample for either male or female determining sperm. These methods are particularly advantageous in the preparation of semen for use in artificial insemination of mammals including, but not limited to, cows, pigs, sheep, goats, humans, camels, horses, deer, alpaca, dogs, cats, rabbits and rodents. Semen used in such methods may be either fresh ejaculate or may have been previously frozen and subsequently thawed.

Methods for separating X- and Y-chromosome bearing sperm include contacting a semen sample with one or more of the binding agents disclosed herein for a period of time sufficient to form a conjugate, or complex, between the sperm and the binding agent, and separating the conjugate(s) from unbound sperm. In one embodiment, magnetic beads, such as paramagnetic microspheres, are coated with a binding agent, such as a binding agent specific for a Y-chromosome specific antigen, and then contacted with a suspension of sperm cells in an appropriate vessel for a period of time sufficient to allow formation of a conjugate of the binding agent and the Y-chromosome specific antigen, thereby linking Y-chromosome bearing sperm to the beads. The sperm containing the Y chromosome are then retained by applying a magnetic force to the vessel, whereas the sperm carrying the X chromosome are easily separated by removing the supernatant from the vessel. Techniques employing magnetic beads for the isolation and/or removal of desired cell types are known in the art and include those described, for example, by Olsaker et al. (Animal Genetics, 24:311-313 (1993)) and in U.S. Pat. Nos. 6,893,881 and 7,078,224.

It will be appreciated that the binding agents disclosed herein may be used in other techniques for separation of desired cell populations well known to those in the art. For example, a native sperm sample may be first exposed to a binding agent disclosed herein, such as an antibody to a Y-chromosome specific antigen, and then to a second antibody that specifically binds to the first antibody, with the second antibody being immobilized on a substrate. Y-chromosome bearing sperm will bind to the first antibody which in turn will bind to the second antibody and become attached to the substrate, thereby separating the Y-chromosome bearing sperm from X-chromosome bearing sperm. Substrates which can be employed in such methods are well known in the art and include, for example, nitrocellulose membranes.

Kits and/or devices for use in the disclosed methods are also provided. In one embodiment, such kits and/or devices include magnetic particles, such as paramagnetic microspheres, coated with, and/or attached to, at least one binding agent for an X- or Y-chromosome specific antigen. The kits and/or devices may be provided in the form of a single use disposable unit that contains sufficient binding agent to process one ejaculate of sperm.

The coated magnetic particles may be employed to separate X or Y-chromosome bearing sperm using known methods, such as those disclosed by Safarik and Safarikova (J. Chromatography, 722:33-53 (1999)). When the binding agent is a mouse monoclonal antibody, for example, beads comprising Protein A coupled to magnetizable polystyrene/iron oxide particles, such as MagaBeads™ Protein A (Cortex Biochen. Inc., San Leandro, Calif., USA) may be employed. The binding agent is cross-linked to the beads using standard chemistry with, for example, a DMP crosslinker (dimethyl primelinidate 2 HCI). Other domains/regions may be employed to link the binding agent to an immobilized support, such as magnetic beads. Conditions for release of the sperm from the magnetic beads are optimized in order to avoid damaging the sperm. For example, a low pH and high glycine concentration may be employed.

In certain embodiments, techniques are employed that both gently release the sperm from binding agent(s) attached to a support (such as magnetic beads) and inactivate the binding agent, thereby preventing its reuse. This can be achieved, for example, by providing a protease recognition site (such as rhino 3c protease) in an exposed part of the framework of the binding agent. Following attachment of the X or Y-chromosome bearing sperm to the immobilized binding agent and removal of the non-bound sperm, protease is employed to cleave the high affinity binding agent, thereby destroying the ability of the binding agent to bind the X or Y-chromosome bearing sperm and releasing the sperm. After cleavage, the sperm can be washed using centrifugation to separate the molecular components from the sperm. The protease recognition site may be partnered with either a disulphide bond or an engineered metal ion binding site (such as calcium, magnesium or zinc) in order to help expose the protease recognition site and/or increase its rate of cleavage by means of reduction or chelation.

In an alternative embodiment, the protease recognition site is provided on a domain/region linking the binding agent to the immobilized support. Addition of protease results in gentle release of the sperm bound to the binding agent.

In yet a further embodiment, chelation and reduction, either alone or in combination, may be employed to release the sperm from the binding agent. For example, chelation of a zinc ion engineered or selected to be integral to the binding agent may be employed to release the binding agent from the sperm. Simultaneously, the binding agent may be attached to the immobilized support by means of a disulphide bond. Reduction would then allow removal of the binding agent from the support. In one method, reduction is required for the chelation, thereby preventing reuse of the system.

Those of skill in the art will appreciate that other methods may be successfully employed for gently releasing the sperm from the immobilized binding agent. For example, biotin could be employed in the site for sperm binding. Subsequent addition of streptavidin would remove the biotin and release the sperm.

In one embodiment, a device employing magnetic beads for sorting one ejaculate has the specifications described in Table 2 below.

TABLE 2
Bovine

| | |
|---|---|
| Sperm in a straw | 1.00E+07 |
| Number of Sexed x-bearing sperm straws/ejaculate | 100 |
| Total sperm required to begin if recover 50% of | 4.00E+09 |
| desired sperm | |
| Number of sperm in typical bull ejaculate | 1.00E+10 |
| Number of ejaculates required | 0.4 |
| Efficiency of cell purification | 0.7 |
| Number of sperm to extract | 1.43E+09 |
| Ratio beads/cell | 6 |
| Required number of beads | 8.57E+09 |
| Bead concentration/ml | 3.00E+10 |
| Volume in this commercial preparation(ml) | 10 |
| Total beads in 10 ml | 3E+11 |
| Volume of MagaBeads ®-Protein A | 2.86E−01 |
| for a single bovine sexing devices (ml) | |
| Volume of MagaBeads ®-Protein A | 3.43E+03 |
| for 12000 bovine sexing devices (ml) | |

Alternative methods for isolating X or Y-chromosome bearing sperm employing a specific binding agent include: (i) agglutination followed by filtration; (ii) non-magnetic beads that have two functional groups, for example, protein A and biotin: the beads are used as described above except that, instead of magnetic separation they are reacted with a surface coated with streptavidin or a similar biotin-binding compound; (iii) immobilization of antibody on a support that allows a column chromatography type approach; and (iv) FACs.

The following examples are offered by way of illustration and not by way of limitation.

### Example 1

### Identification of Candidate Genes by Bioinformatics

The publicly available bovine genome (available on the Ensembl website; originally released on Aug. 14, 2006; updated version released in February 2007) together with the publicly available human genome, was used in a genomics based method to identify differences on the surface of sexed semen. Specifically, candidate genes were selected using the Ensembl Biomart tool (available on the Ensembl website) and the following strategy:

1) identify bovine orthologues of human X chromosome genes that have a transmembrane domain using Biomart and check by manual analysis;

2) identify genes in the bovine genome that are present on the X chromosome and have a transmembrane domain by Biomart and check by manual analysis (no sequenced bovine Y chromosome); and

3) identify bovine orthologues of human Y chromosome genes that have a transmembrane domain using Biomart and check by manual analysis (one bovine gene was included that could have moved to the X chromosome in bovine).

After removing redundant hits, a total of 216 candidate genes were identified.

### Example 2

### Prioritization of Candidate Genes Based Upon Expression Levels

Each candidate gene identified in Example 1 was examined to see if there were splice variants and if so, an exon common to all transcripts was selected. If no suitable exons were present, an exon unique to each transcript was selected for primer design. Exons were employed for primer design, instead of across introns, to allow all the primers to be verified on genomic DNA. Control primers were also designed to ensure the absence of genomic DNA in the cDNA. Primers were designed for real-time PCR using the Primer3 software (available on-line from SourceForge) with a product size of 80-150 bp. All primers were checked using the Blast software to confirm that they could not prime elsewhere in the genome (i.e. that at least the 3′ end base of the primer could not match). The designed primers were then employed in reverse transcription PCR studies to analyse expression of the candidate genes in bovine testis tissue cDNA and bovine genomic DNA.

Of the initial 216 candidate genes, 136 were shown to be expressed in bovine testis tissue. These genes were then prioritized by applying a criteria based on expression and subcellular location as described below.

Round spermatids are developing sperm cells that have undergone meiosis and, unlike mature sperm, transcribe RNA. Round spermatids (RS) differentiate into spermatozoa (mature sperm) without cell division and thus represent a good candidate to identify expressed genes in sperm.

The feasibility of this approach was demonstrated by showing a high correlation between proteins present on the surface of murine sperm and expression of mRNA in murine RS. This comparison was made using data from two high quality publications. The first publication (Stein et al., 2006) assembled 82 proteins present on sperm surface membranes by using a combination of membrane purification and mass spectrometry. The mRNA expression of these 82 proteins was then examined in murine RS provided by the second publication (Shima et al., 2004). Of the 82 genes, there was data for 71 genes and, of these 71, 67 expressed gene at the RNA level (94%). This result demonstrates that mRNA expression in RS is a good indirect measure of sperm proteins.

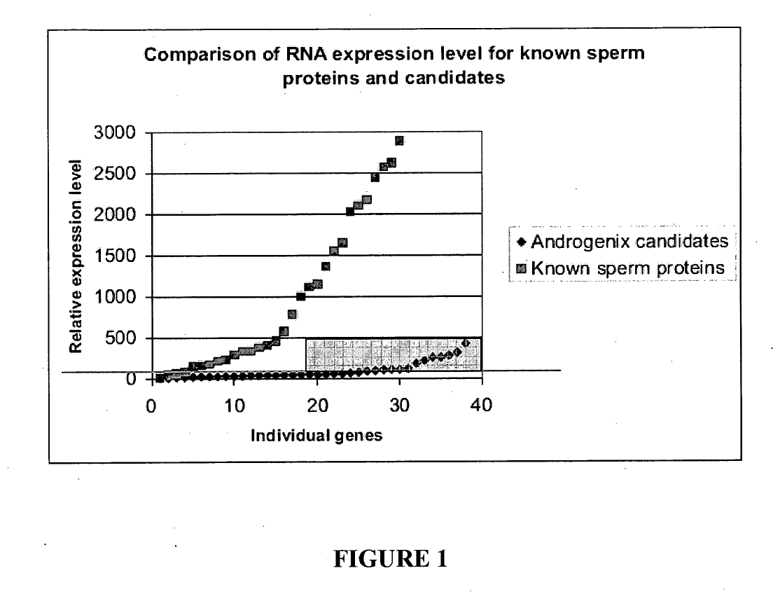

The murine data sets were mined further to look at the relative amount of RNA expression in the RS for the gene products known to be present on the cell surface and compared to the RNA expression level for the murine orthologues of the candidates. The results of this analysis, which are shown in FIG. 1, demonstrate that the candidate genes are generally expressed at a much lower level than the random selection of known sperm proteins (approximately 30 of the 71 genes for which there was expression data). These results potentially explain why researchers have so far been unable to discover surface differences between sperm that bear the X or Y chromosome, and indicate that such differences will require very sensitive tools to detect and exploit.

These results also allowed the candidate genes to be prioritized based upon relative expression amount. Apart from one gene, the proteins detected in Stein et al. (Ibid had an RNA expression level of greater than 40, thus this number was taken as a threshold to focus on the best candidates. The highest priority candidate genes (indicated by the box in FIG. 1) all have a relative expression level of 40 or above, based on the murine orthologue. An examination of other proteins known to be detected by antibodies on sperm indicated a range of expression levels from 9-1000 (see Table 3).

TABLE 3
proteins detected on sperm and their mRNA expression in murine RS

| | mRNA | | |
|---|---|---|---|
| | expression | | |
| | in | | |
| Gene | RS (NCBI | Species sperm | |
| name | GEO) | protein detected in | Reference |
| Ph20 | 1000 | bovine | PMID: 15892045; Morin et |
| spam1 | | | al. Mol. Reprod. Dev. 71: |
| | | | 523-534 (2005) |
| Csf2ra | 65 | human bovine | PMID: 11169747; |
| | | | Zambrano et al. J. Cell |
| | | | Biochem. 80: 625-634 (2001) |
| Csf2rb1 | 20 | human bovine | PMID: 11169747; |
| | | | Zambrano et al. J. Cell |
| | | | Biochem. 80: 625-634 (2001) |
| Trpc1 | 40 | human/mouse | PMID: 12706821; |
| | | | Castellano et al. FEBS Lett. |
| | | | 541: 69-74 (2003); PMID: |
| | | | 11734218; Trevino et al. |
| | | | FEBS Lett. 509: 119-125 |
| | | | (2001) |
| Cnr2 | 14 | boar | PMID: 16144868; |
| | | | Maccarrone et al., J. Cell |
| | | | Sci. 118: 4393-4404 (2005) |
| Cnr1 | 50 | boar | PMID: 16144868; |
| | | | Maccarrone et al., J. Cell |
| | | | Sci. 118: 4393-4404 (2005) |
| drd2 | 47 | rat, mouse, human | PMID: 16924680; Otth et |
| | | and bull | al., J. Cell Biochem. |
| | | | 100: 141-150 (2007) |
| CCR5 | 9 | human | PMID: 16174786; |
| | | | Muciaccia et al., Faseb J. |
| | | | 19: 2048-2050 (2005) |
| il6st | 50 | human | PMID: 16728717; Cai et al. |
| | | | J. Androl. 27: 645-652 |
| | | | (2006) |

PMID = unique Public Medline identifier

### Example 3

### Prioritization of Candidate Genes Based Upon Subcellular Localization

The low level expression of the candidate genes in round spermatids suggests that, if a candidate resides solely on a membrane other than the cell surface, then these candidates should be given a lower priority. The reason for this action is that, as the candidates already have low expression, this coupled with only a small percentage of the protein being on the surface would make the candidate very difficult to detect. The candidate genes, or their orthologues in other species, were therefore examined to determine the subcellular location of the gene product. If evidence was available that the protein was on a membrane system other than the cell surface, this candidate was given a lower priority. This data was combined with the round spermatid expression data to generate four gene classes of differing priority, with Class I being the highest priority. Each of the Class I bovine candidate genes, which are identified in Table 4 below, have the following properties:
the murine orthologue gene is expressed above the threshold level (see above) in mouse round spermatids;
the gene is expressed in bull testis tissue;
the gene products are very likely to reside in a cell membrane; and the gene products are either known to reside on the cell surface or there is no evidence that the gene products do not reside on the cell surface.

TABLE 4
Class I candidate bovine genes

| | | Amino Acid | DNA SEQ ID |
|---|---|---|---|
| Bovine Gene Ensembl ID | Gene Name | SEQ ID NO: | NO: |
| ENSBTAG00000000520 | ATP2B3 | 1 | 22 |
| ENSBTAG00000005616 | BRS3 | 2 | 23 |
| ENSBTAG00000006296 | Unknown1 | 3 | 24 |
| ENSBTAG00000006818 | FAM11A | 4, 138 | 25, 137 |
| ENSBTAG00000007859 | VSIG1 | 5 | 26 |
| ENSBTAG00000009959 | SLC6A8 | 6, 140 | 27, 139 |
| ENSBTAG00000010018 | ATP7A | 7, 142 | 28, 141 |
| ENSBTAG00000012718 | XK | 8, 144 | 29, 143 |
| ENSBTAG00000013462 | L1CAM | 9 | 30 |
| ENSBTAG00000014798 | CXCR3 | 10 | 31 |
| ENSBTAG00000016484 | ATP11C | 11, 146 | 32, 145 |
| ENSBTAG00000017801 | ATP6AP2 | 12, 148 | 33, 147 |
| ENSBTAG00000019552 | PGRMC1 | 13 | 34 |
| ENSBTAG00000035134 | Unknown2 | 14, 150 | 35, 149 |
| ENSBTAG00000001338 | CCR3 | 15 | 36 |
| ENSBTAG00000002923 | CX3CR1 | 16, 152 | 37, 151 |
| ENSBTAG00000005781 | unknown 3 | 17, 154 | 38, 153 |
| ENSBTAG00000015801 | EFNB1 | 18 | 39 |
| ENSBTAG00000020826 | CHIC1 | 19 | 40 |
| ENSBTAG00000032501 | unknown 4 | 20, 156 | 41, 155 |
| ENSBTAG00000034045 | FMR1NB | 21, 158 | 42, 157 |
| ENSBTAG00000014533 | Kel | 160 | 159 |
| ENSBTAG00000035195 | Unknown 5 | 162 | 161 |
| ENSBTAG00000035944 | Unknown 6 | 164 | 163 |

Based on comparisons with various mammalian orthologues, certain of the sequences provided in SEQ ID NO: 1-42 were found to have potential prediction errors. Amended, more accurate, sequences are provided in SEQ ID NO: 137-158.

Human genes corresponding to the candidate bovine genes are identified in Table 5.

TABLE 5
Class I candidate human genes

| | | | Human | Human |
|---|---|---|---|---|
| | | | DNA | Amino |
| Bovine Gene | Human Gene | Human Gene | SEQ ID | Acid SEQ |
| Ensembl ID | Ensembl ID | Name | NO: | ID NO: |
| ENSBTAG00000000520 | ENSG00000067842 | ATP2B3 | 122, 120, | 77, 73, 86, |
| | | PMCA3 | 133, 131, | 84, 80, 88 |
| | | | 127, 135 | |
| ENSBTAG00000001338 | ENSG00000183625 | CCR3 | 95 | 48 |
| ENSBTAG00000002923 | ENSG00000168329 | CX3CR1 | 107 | 60 |
| ENSBTAG00000005616 | ENSG00000102239 | BRS3 | 96 | 49 |
| ENSBTAG00000005718 | ENSG00000124103 | unknown 3 | 97, 94, 91 | 50, 47, 44 |
| ENSBTAG00000006296 | ENSG00000160131 | Unknown1 | 118, 123 | 71, 76 |
| ENSBTAG00000006818 | ENSG00000155984 | FAM11A | 117 | 80 |
| ENSBTAG00000007859 | ENSG00000101842 | VSIG1 | 90 | 43 |
| ENSBTAG0000009959 | ENSG00000130821 | SLC6A8 CTI | 119, 121, | 72, 74, 75 |
| | | | 124 | |
| ENSBTAG00000010018 | ENSG00000165240 | ATP7A | 110, 100, | 63, 53, 54, |
| | | | 101, 93, | 46, 59 |
| | | | 106 | |
| ENSBTAG00000014798 | ENSG00000186810 | CXCR3 | 108, 112, | 61, 65, 62 |
| | | | 109 | |
| ENSBTAG00000015801 | ENSG00000090776 | EFNB1 | 92 | 45 |
| ENSBTAG00000016484 | ENSG00000101974 | ATP11C | 111, 126, | 64, 79, 68, |
| | | | 115, 114, | 67, 78 |
| | | | 125 | |
| ENSBTAG00000017801 | ENSG00000182220 | Renin | 102 | 55 |
| | | receptor | | |
| ENSBTAG00000019552 | ENSG00000101856 | PGRMCI | 105 | 58 |
| ENSBTAG00000020826 | ENSG00000204116 | CHIC1 | 99, 98 | 52, 51 |
| ENSBTAG00000034045 | ENSG00000176988 | FMR1NB | 113, 116 | 66, 69 |
| ENSBTAG00000035134 | ENSG00000189118 | Unknown2 | 104 | 57 |
| ENSBTAG00000014533 | ENSG00000197993 | Kel | 202 | 201 |

Equine genes corresponding to the candidate bovine genes are identified in Table 6.

TABLE 6
Class I candidate equine genes

| | | | Equine | Equine |
|---|---|---|---|---|
| | | | DNA | Amino |
| Bovine Gene | Equine Gene | Equine | SEQ ID | Acid SEQ |
| Ensembl ID | Ensembl ID | Gene name | NO: | ID NO: |
| ENSBTAG00000001338 | ENSECAG00000001282 | CCR3 | 165 | 183 |
| ENSBTAG00000013462 | ENSECAG00000002810 | L1CAM | 166 | 184 |
| ENSBTAG00000002923 | ENSECAG00000004442 | CX3CR1 | 167 | 185 |
| ENSBTAG00000035134 | ENSECAG00000005133 | | 168 | 186 |
| ENSBTAG00000016484 | ENSECAG00000005393 | ATP11C | 169 | 187 |
| ENSBTAG00000005616 | ENSECAG00000008806 | BRS3 | 170 | 188 |
| ENSBTAG00000006818 | ENSECAG00000009399 | Fam11a | 171 | 189 |
| ENSBTAG00000019552 | ENSECAG00000009619 | PGRMC1 | 172 | 190 |
| ENSBTAG00000014533 | ENSECAG00000010525 | KEL | 173 | 191 |
| ENSBTAG00000015801 | ENSECAG00000012319 | EFNB1 | 174 | 192 |
| ENSBTAG00000009959 | ENSECAG00000013965 | | 175 | 193 |
| ENSBTAG00000012718 | ENSECAG00000014332 | XK | 176 | 194 |
| ENSBTAG00000020826 | ENSECAG00000016317 | CHIC1 | 177 | 195 |
| ENSBTAG00000010018 | ENSECAG00000016767 | ATP7A | 178 | 196 |
| ENSBTAG00000007859 | ENSECAG00000018968 | VSIG1 | 179 | 197 |
| ENSBTAG00000017801 | ENSECAG00000019889 | ATP6AP2 | 180 | 198 |
| ENSBTAG00000000520 | ENSECAG00000023490 | ATP2B3 | 181 | 199 |
| ENSBTAG00000014798 | ENSECAG00000023587 | CXCR3 | 182 | 200 |

Apart from three genes, all the Class I candidate genes were selected from the X chromosome of either human, cow or horse. Two exceptions, which are both chemokine receptors, were from two papers where the authors observed that, when staining sperm with antibodies specific for the chemokine receptors (CCR3 & CX3CR1), only 50% of the sperm stained (Muciaccia et al., Faseb J. 19:2048-2050 (2005); Zhang et al., Hum. Reprod. 19:409-414 (2004)). Both these chemokine receptors are tightly clustered on bovine chromosome 22 and, upon inspection of their promoter regions, it is possible that GATA-1, an X-encoded transcription factor, may bind and control their expression (DeVries et al., J. Biol. Chem. 278:11985-11994 (2003); Garin et al., Biochem. J. 368:753-760 (2002); Vijh et al., Genomics 80:86-95 (2002); Zimmermann et al., Blood 96:2346-2354 (2000)). The other exception kel is the disulphide linked partner of the XK protein (Lee et al., Semin. Hematol. 37:113-121 (2000); Russo et al., Biochim. Biophys. Acta 1461:10-18 (1999); Russo et al., J. Biol. Chem. 273:13950-13956 (1998)).

The mouse orthologue of one of the candidate genes disclosed herein (pgrmcl; mouse protein ENSMUSG0000006373; bovine protein ENSBTAG00000019552, SEQ ID NO: 13) has been shown to be present on sperm membrane in a proteomic study by Stein et al. (Proteomics 6:3533-3543 (2006); see also, Baker et al., Proteomics 8:1720-1730 (2008)). In addition, the mouse orthologue of another Class I candidate antigen identified using the methods described herein (mouse protein ENSMUSG00000031130: bovine protein ENSBTAG0000005616; SEQ ID NO: 2) has been shown to be expressed on developing sperm (Fathi et al., J. Biol. Chem. 268:5979-5984 1993)).

### Example 4

### Generation of Antibody Detection Reagents and Testing of Expressed Genes for Presence on the Sperm Surface

The availability of the Class I candidate genes enabled them to be examined closely for potential errors in their predicted sequence. Based upon comparison with other mammalian orthologues, several candidate genes were discovered to have potential prediction errors and new gene models were created and tested by cloning either portions of the cDNA or the entire open reading frame and sequencing these regions. The configuration of the Class I candidate proteins in the membrane was either determined from the literature or modelled, and used in the selection of peptides for antibody generation.

For each of the Class I candidate genes, a specific strategy was developed to show the protein is present on the surface of sperm and verify that the gene product is specific for either X- or Y-chromosome bearing sperm. These strategies, which are shown in Table 7 below, include using bovine and/or human sperm, together with obtaining antibodies from a combination of commercially available and/or generation of antibodies through two different peptide-based approaches and, for three of the candidate genes, expression and purification of the recombinant proteins.

TABLE 7

| SEQ | | | |
|---|---|---|---|
| ID | | Membrane | |
| NO: | Gene name | type | Strategy |
| 1 | ATP2B3 | 10TM | Commercial antibodies and Siscapa |
| | PMCA3 | | approach |
| 15 | CCR3 | 7TM | Commercial antibodies |
| 16 | CX3CR1 | 7TM | Commercial antibodies |
| 2 | BRS3 | 7TM | Commercial antibodies plus Siscapa |
| | | | approach |
| 17 | unknown 3 | Type I | Peptide antibodies and Siscapa approach |
| 3 | Unknown 1 | 2TM | Peptide antibodies and Siscapa approach |
| 4 | FAM11A | 8TM | Peptide antibodies and Siscapa approach |
| 5 | VSIG1 | Type I | Express extracellular domain and |
| | | | generate antibodies, and also Siscapa |
| | | | approach |
| 6 | SLC6A8 | 12TM | Peptide antibodies, one commercial |
| | CT1 | | Antibody and Siscapa approach |
| 7 | ATP7A | 8TM | Commercial antibodies and Siscapa |
| | | | approach |
| 8 | XK | 10TM | Commercial antibodies (both XK and |
| | | | Kell) and Siscapa approach |
| 9 | L1CAM | Type I | Commercial antibodies and Siscapa |
| | | | approach |
| 10 | CXCR3 | 7TM | Commercial antibodies |
| 18 | EFNB1 | Type I | Express extracellular domain and |
| | | | generate antibodies, and also Siscapa |
| | | | approach |
| 11 | ATP11C | 10TM | Peptide antibodies, also buy one peptide |
| | | | human antibody and Siscapa |
| 12 | Renin | Type I | Express extracellular domain and |
| | receptor | | generate antibodies and also Siscapa |
| | | | approach |
| 13 | PGRMC1 | 1TM | Peptide antibodies one commercial |
| | | | antibody and Siscapa approach |
| 19 | CHIC1 | 1TM | Peptide antibodies and Siscapa approach |
| 20 | unknown 4 | Type I | Peptide antibodies and Siscapa approach |
| 21 | FMR1NB | 2TM | Peptide antibodies, one commercial |
| | | | antibody and Siscapa approach |
| 14 | Unknown2 | 4TM | Peptide antibodies only |

Peptide-generated antibodies often have a range of titres and do not necessarily recognize native proteins or proteins denatured on SDS-PAGE gels. Additionally, integral membrane proteins (the majority of the Class I candidates) are often difficult to solubilize and thus get into PAGE gel systems (Peirce et al., Mol. Cell Proteomics 3:56-65 (2004); Santoni et al., Electrophoresis 21:3329-3344 (2000)). Our solution to these problems was the following: generate multiple peptide-antibodies per protein and use a variety of detection techniques for these antibodies, such as direct cell surface binding (Flow cytometry), cell lysis assays, antibody sperm capture, Western blotting and/or immunohistochemistry of fixed sperm cells. In another strategy that overcomes some of the problems of peptide-generated antibodies, mass spectrometry is used to detect the candidate sperm surface proteins.

These experiments have two goals: first determine if any of the candidate genes are present in the sperm; and second, if present, determine whether the candidate is on the plasma membrane, determine distribution across sperm bearing either the X or Y chromosome and confirm the identity of bound species. To achieve a high assay throughput, where possible a robotic station in conjunction with 96/384 well plates was used to setup and perform the assays.

a) Production of Antibodies and/or Antisera

Peptides for production of antibodies to the Class I candidate antigens of SEQ ID NO: 1-9, 11-14 and 17-21 were designed using the strategies described below. Two design strategies were followed for peptide selection/design. In the first strategy, standard peptide design rules were applied to design peptides that bind preferentially to surface exposed epitopes, however if insufficient surface epitopes were available cytoplasmic epitopes were used. Briefly, the approach for designing peptides was as follows: chose the N-terminus, C-terminus and small loops connecting transmembrane domains (that had been mapped on the sequence; predicted signal sequences were removed from the sequence for peptide selection); and choose a sequence that had a suitable hydrophilicity (−0.5 to 0.5), did not begin with glutamic acid or glutamine, did not have any cys residues, did not have a likely glycosylation site and was not closely related to other proteins. All peptides have a linker usually at the c-terminus (GSGC) to enable specific coupling to the carrier protein, ELISA plate and/or agarose for affinity purification of the antisera. However, for peptides that were at the very C-terminus of a protein, the linker CGSG was added to the N-terminus. In the second strategy, peptides were designed for use in the SISCAPA technique according to the methodology of Anderson et al. (J. Proteome Res. 3:235-244 (2004)). Essentially, this technique is an ELISA with the detection phase being mass spectrometry.

Following peptide design and production, peptides were conjugated to the carrier KLH and employed to immunize rabbits, using standard techniques for the production of antisera. Peptides may also be conjugated to a second carrier to act as a positive control in various assays. Alternatively, ELISA plates having a covalently attached malemide (cys reactive) group may be employed. For each candidate gene, two peptides were simultaneously immunized into a rabbit and two rabbits were immunized for each pair of peptides. This approach is efficient in its use of animals, maximises the likelihood of obtaining antibodies with the required activity and, with affinity purification of the antibody, provides monospecific antisera (Larsson et al., J. Immunol. Methods 315:110-120 (2006); Uhlen and Ponten, Mol. Cell. Proteomics 4:384-393. (2005)).

Antisera were tested for recognition of the immunizing peptide by ELISA. In brief, purified peptide was attached specifically to the ELISA plate through the free sulphydral group (cys residue) on each peptide. The free thiol group was reacted with ELISA microplates that have a maleimide surface (Corning) thus allowing irreversible binding of the peptide via the thiol group. Following peptide binding, the antisera (both pre-immune and final bleed) was titrated against the peptide. Subsequently HRP-conjugated anti-rabbit antibodies were added and the signal developed using OPD.

The results indicated that for the 54 peptides employed in the immunizations, antisera that had a specific peptide binding titre of 0.001 or less was achieved for 25 peptides. The antiserum with a titre less than 0.001 was purified on columns with the peptide specifically attached through the free thiol group by standard techniques. Desalted antibodies were used in subsequent assays.

### b) Binding of Antibodies to Candidate Gene Products

The specificity of antibodies for candidate gene products was determined as follows. The genes for the majority of candidates were cloned to enable their use as positive controls. The genes were either purchased or cloned and then transferred to the Invitrogen expression vector pcDNA™3.2/V5-DEST. These plasmids were used to transiently transfect HEK 293T cells by the calcium phosphate method. After 48-72 hours, the cells were either scraped from the culture dishes for use in flow cytometry studies or used directly to create whole cell lysates.

The ability to compare HEK cells mock transfected or transfected with the appropriate expression vector and subsequent flow cytometry analysis with the candidate antibodies allowed verification that the antibodies were specific for the candidate gene products. These results are summarized in the Table 8 below.

TABLE 8

| | Identification of antibodies that show |
|---|---|
| | specific binding to transiently transfected |
| Candidate | HEK293T cells by flow cytometry |
| CCR3 | (SC32777; SC7897; MAB155) |
| CX3CR1 | (SC20432; SC30030) |
| BRS3 | (SC33404) |
| FAM11A | GN21352) |
| VSIG1 | (VSIG) |
| XK | SC50201; SC50202) |
| Kel | (IBGRL9440; IBGRL9441; AF1914) |
| L1CAM | (SC33686; SC31034; SC53386; SC15326) |
| CXCR3 | (SC57076; SC9900) |

c) Assays for Agents that Bind the Candidate Gene Products

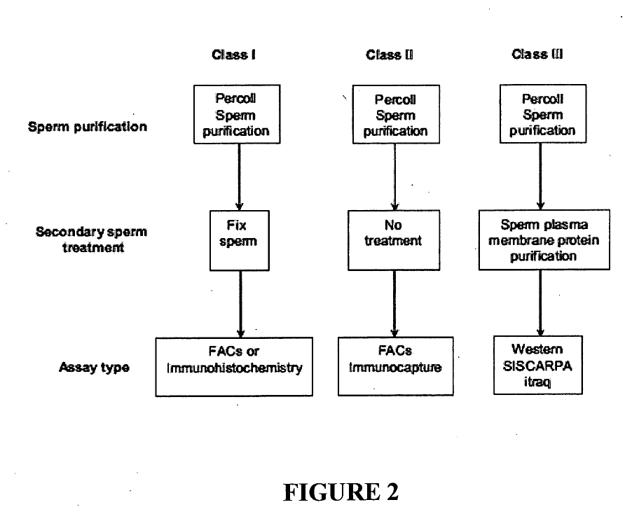

As the nature of binding of peptide-generated antibodies to the target protein is hard to predict (i.e. whether the antibody will recognize the native protein and/or denatured versions), a variety of assays are used. Assays to examine binding of antibodies or other agents to the candidate gene products include the following as classified by the starting material and the assay used (see FIG. 2):
Class I assays: Intact sperm assays using either flow cytometry, cell lysis and/or immunopurification;
Class II assays: Fixed sperm assays using either immunohistochemistry type approaches and/or a flow cytometry readout; and
Class III assays: Sperm surface membrane protein preparation followed by Western blotting, SISCAPA and/or iTraq approach.

### Class I Assays

These assays use living sperm either fresh or thawed from aliquots frozen in liquid nitrogen. Bovine sperm are purified by Percoll™ gradients to produce a viable, highly motile, morphologically normal and fertilizable population of sperm (Samardzija et al., Anim. Reprod. Sci. 91:237-247 (2006); Trentalance and Beorlegui, Andrologia 34:397-403 (2002)). This procedure has been used previously on both fresh and frozen sperm. The Class I assays utilize the antibodies/binding agents described above and detection comprises flow cytometry and Alexa Fluor conjugated secondary antibodies (Invitrogen Corp., Carlsbad, Calif.) as a reporter system, immunocapture of sperm with paramagnetic beads, and also immunoprecipitation followed by detection of the released trypsin digested proteins by mass spectrometry.

### Class II Assays

The rationale for using immunohistochemistry is that fixation can alter protein epitopes and may make certain epitopes available that are not in the native protein. Before use, bovine sperm are purified on Percoll™ gradients and then fixed with a range (3-4) of different fixatives. Again the antibodies/binding agents described above are used and the readout for binding is flow cytometry or an ELISA plate-based format.

### Class III Assays

The class III assays are likely to be the most sensitive for detection of low abundance antigens. Again bovine sperm are purified on Percoll™ gradients and sperm plasma membrane protein fractions are then prepared by two different techniques. The first method biotinylates the surface of intact sperm, with the plasma membrane proteins subsequently being isolated on nutra-avidin and used in various assays (Zhao et al., Anal. Chem. 76:1817-1823 (2004)). A second method for plasma membrane protein preparation involving more traditional nitrogen cavitation/sedimentation and detergent solubilization (Lalancette et al., Biol. Reprod. 65:628-636 (2006)) can also be used. Two different techniques for membrane protein isolation are used as all methods have some selectivity towards isolation of different proteins.

After the enrichment of the sperm plasma membrane the sample is used in the following three assays:

(i) Western Blotting

The key issue for western blotting is getting sufficient amount of the enriched plasma membrane protein into the gel for PAGE while still allowing the gel to resolve well and provide sufficient sensitivity. Before loading the plasma membrane enriched sample onto the PAGE gel, further simple fractionation may be used, such as a simple size cut-off using spin columns e.g. retain material above 10 Kd. Detergents/phase separation may also be used to select for membrane proteins of certain types, for example single-pass or multi-pass (Santoni et al., Electrophoresis 21:3329-3344 (2000)). Overall, the aim is to create knowledge-based enrichment (using the candidate gene information) without creation of additional samples, thus candidate genes may be grouped for various treatments. In one embodiment, proteins are first immunoprecipitated with several antibodies and the captured proteins then identified using western blotting.

(ii) SISCAPA

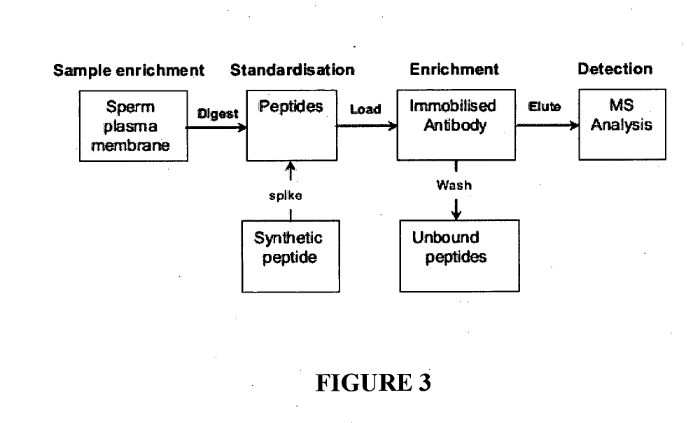

The outline of the SISCAPA technique is shown in FIG. 3. The major difference from the standard technique is that a different starting material will be used, namely sperm plasma membrane as opposed to human plasma proteins, and the isotopically labelled peptide will be omitted.

The SISCAPA technique was designed to specifically identify and quantify proteins in human plasma that change with various metabolic or disease states (Anderson et al., 2004, Ibid). This powerful technology has several advantages:
Uses antibodies to enrich the sample peptides, thus reducing the complexity for mass spectrometry analysis;
Antibodies raised against peptides almost always recognise the peptide, unlike the parent protein;
Spiked peptides (isotopically labelled in Anderson's case) allow the mass spectrometer to unambiguously identify the peptide and also quantitate the endogenous protein. In the current studies, the SISCAPA method is performed using the same peptide as used for immunization instead of the istopically labelled peptide. This peptide acts as a standard to determine the flight characteristics of the peptide in the mass spectrometer. The peptides employed in the current studies have a GSGC linker, however this will be after a basic residue and thus digesting the peptide with trypsin will provide the exact peptide as an internal control for the mass spectrometer;
The trypsin digestion of the starting sample also has significant advantages, particularly for membrane proteins where digesting to peptides enables solubilization and separation, tasks that are considerably more difficult with the hydrophobic parent proteins; and
The amount of sample applied to the antibodies is not limited, thus enabling a very large number of sperm cell plasma membranes (>10⁸ cell equivalents) are to be passed over the antibodies, which in turn provides the technique with potentially very high sensitivity.

(iii) iTRAQ™

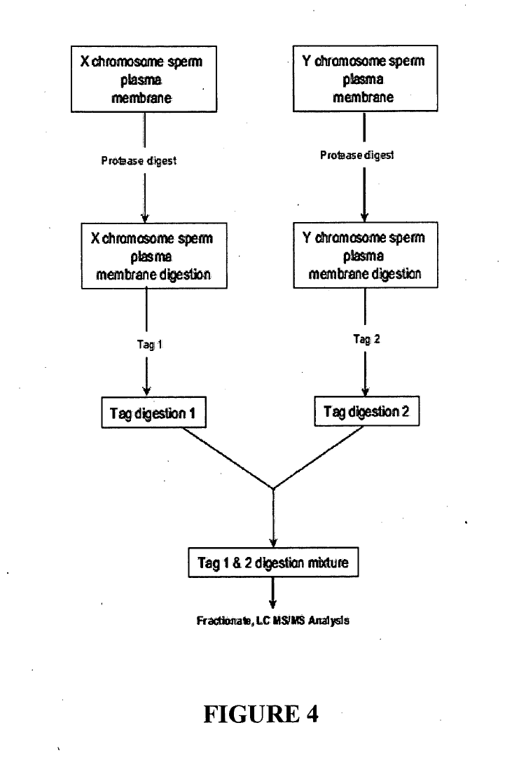

Applied Biosystems iTRAQ™ reagents are a multiplexed set of four isobaric reagents which are amine specific and yield labelled peptides which are identical in mass and hence also identical in single MS mode, but which produce strong, diagnostic, low-mass MS/MS signature ions, allowing for quantitation of up to four different samples simultaneously. Protein identification is simplified by improved fragmentation patterns, with no signal splitting in either the MS or MS/MS modes and the complexity of MS and MS/MS data is not increased by mixing multiple proteome samples together. The current studies employ the iTRAQ™ technology as depicted in FIG. 4. In contrast to other techniques employed in the current studies, the sperm are first sorted by flow cytometer into two populations bearing either the X or Y chromosome. These sorted samples are then used with the iTRAQ™ reagents.

### Example 5

### Antibody Binding to Sperm Cells and Analysis By Flow Cytometry

For ten of the candidate genes disclosed herein, antibodies specific for the candidate proteins were shown to bind sperm from either human, bovine or both using flow cytometry as follows.

Fresh sperm were purified by centrifugation on Percoll™ (GE Healthcare) discontinuous density layers. Following washing, visual microscopic inspection of sperm showed an essentially pure population of sperm. Human sperm derived from a single ejaculate had a range of concentration (20-60 x10⁶/ml) with a total count of 40−120×10⁶ sperm, motility as assessed visually averaged >60%. Bovine sperm average concentration was 1.5×10⁹/ml with a total count of approximately 10×10⁹ sperm, motility as assessed visually averaged greater than 70%. The Invitrogen LIVE/DEAD Sperm Viability Kit (SYBR-14/propidium iodide) was used to assess viability of purified sperm. Generally sperm showed greater than 80% viability as assessed by the Sperm Viability Kit. In addition, analysis by Lysotracker™ (Invitrogen) showed that less than 20% of sperm were acrosome reacted and that this fraction equated to the dead population from the sperm viability analysis.

Antibody staining of purified sperm was performed by standard techniques. Briefly, purified sperm were incubated with their primary antibodies, washed and labeled with Alexa Fluor 488™ conjugated secondary antibodies. Before analysis, cells were also stained with propidium iodide. Dead sperm were excluded by propidium iodide staining and for each analysis 30,000 events were collected in a Becton-Dickinson FACScalibur.

The results of these studies are summarized in Tables 9 and 10 below. Where specific binding of candidate antibodies to sperm was shown, this was also achieved for sperm samples from more than one individual.

TABLE 9

| | Number of antibodies showing | Identification of |
|---|---|---|
| | binding to human sperm by flow | antibodies that show |
| | cytometry as a proportion of | binding to human sperm |
| Candidate | those tried | by flow cytometry |
| CCR3 | 2/3 | SC7897; MAB155 |
| CX3CR1 | 2/3 | SC20432; SC30030 |
| BRS3 | 1/2 | SC33404 |
| FAM11A | 1/2 | GN21352 |
| VSIG1 | 0/1 | |
| XK | 2/3 | SC50201; SC50202 |
| Kel | 3/3 | IBGRL9440; IBGRL9441; |
| | | AF1914 |
| L1CAM | 3/5 | SC31034; SC53386; |
| | | SC15326 |
| CXCR3 | 2/4 | SC57076; SC9900 |
| FMR1NB | 1/1 | FMR1NB |

TABLE 10

| | Number of antibodies showing | |
|---|---|---|
| | binding to bovine sperm by flow | Identification of antibodies |
| | cytometry as a proportion of | that show binding to bovine |
| Candidate | those tried | sperm by flow cytometry |
| CCR3 | 1/3 | SC7897 |
| CX3CR1 | 1/3 | SC30030 |
| BRS3 | 1/2 | SC33404 |
| FAM11A | 1/2 | GN21352 |
| VSIG1 | 1/1 | VSIG |
| XK | 2/3 | SC50201; SC50202 |
| Kel | 3/3 | IBGRL9440; IBGRL9441; |
| | | AF1914 |
| L1CAM | 2/5 | SC53386; SC15326 |
| CXCR3 | 2/4 | SC57076; SC9900 |
| FMR1NB | 1/1 | FMR1NB |

The percentage of sperm cells showing specific binding varied depending upon the antibody as shown in Table 11 below.

TABLE 11

| | | Sperm | |
|---|---|---|---|
| | | species | Sperm cells |
| | | used for | showing |
| | | binding | specific |
| Antigen | Class | experiment | binding (%) |
| XK; ENSG00000047597 | Candidate | Human | 24.2 |
| CCR3; ENSG00000183625 | Candidate | Human | 31.7 |
| BRS3; ENSG00000102239 | Candidate | Human | 10.7 |
| CX3CR1; ENSG00000168329 | Candidate | Human | 10.5 |
| CXCR3; ENSG00000186810 | Candidate | Human | 11.9 |
| FMR1NB; ENSG00000176988 | Candidate | Human | 29.7 |
| FAM11A; | Candidate | Bovine | 8.4 |
| ENSBTAG00000006818 | | | |
| KEL; ENSG00000197993 | Candidate | Human | 14.4 |
| L1CAM; ENSG00000198910 | Candidate | Human | 19.0 |
| VSIG1; | Candidate | Bovine | 5.2 |
| ENSBTAG00000007859 | | | |
| CD55 | Control | Human | 71.0 |

For the examples showing a higher percentage of binding, namely XK, CCR3, FMR1NB and L1CAM, there is clear evidence of antibody binding in a bimodal distribution, a first peak coincident with the secondary antibody only peak and a second distribution with approx. 1-100 fold more fluorescence. The antibodies that displayed a lower percentage of cells binding showed a skewing of the fluorescence distribution (relative to the secondary only antibody peak) with 1-10 fold more fluorescence. These results contrast with the data obtained from using anti-CD55 antisera as an antibody known to bind to the sperm surface. This antibody specifically bound to 71% of the sperm, however there was only a uni-modal binding distribution for both the secondary antibody alone and also the primary and secondary antibody together, although for the latter binding the whole peak shifted due to the greater fluorescence. As antibody binding is a function of number of binding sites available and the affinity of the antibody, the less than 50% of cells binding antibody may indicate that there is very low expression of molecules on the sperm surface (below detection with the reagents used) and/or that not all sperm bear the candidate antigens.

In general more antibodies bound to human sperm than bovine sperm. This result would be expected as the majority of the antibodies were generated to human proteins. The candidate proteins are in general highly conserved between human and bovine, however small changes in amino acid sequence (depending upon the epitope) may lower the affinity of the antibody for the protein.

### Example 6

### Antibody Binding to Sperm Cell Preparations and Analysis by Western BLOT

The ability of antibodies to candidate gene products to bind sperm cell preparations was examined by Western blot as follows.

Purified sperm were subjected to sonication, nuclei were removed by centrifugation and the total membrane fraction isolated by ultra-centrifugation. Protein from the membrane fraction were separated on 8% Bis-Tris polyacrylamide gels (Invitrogen) and transferred to nitrocellulose membrane (NC; Invitrogen i-Blot). The NC membrane was blocked with non-fat milk, incubated with a primary antibody specific for the protein of interest and then with Horseradish-peroxidase conjugated secondary antibodies. The blot was developed with chemiluminescent ECL Western blotting substrate and signals detected using a LAS-3000 imaging system (Fuji).

The antibody AF914 specific for human kel was used in a western blot to detect a band that ran just below the 100 kD. The band appeared in the lane loaded with a membrane preparation from 0.8×10⁸ human sperm. An almost identical size band was also western blot loaded with whole cell lysates from HEK cells transfected with the pCDNA vector expressing the human kel gene. In contrast whole cell lysates from untransfected HEK cells did not show antibody specific binding.

An antibody made in rabbits to the recombinant extracellular domain of the bovine EFBN1 gene was used to probe western blots of bovine sperm. In these experiments membrane preparations from 1×10⁹ human sperm were run on SDS-PAGE, blotted to nitrocellulose and the anti-EFBN1 antibody used to detect the EFBN1 protein. The sperm membrane lane showed a bans present at 50 kd. An identical lane probed with the pre-immune antiserum did not show a similar band. The human EFBN1 that is 96% identical at the amino acid level has been shown to run at 50 kD (PMID 17567680).

### Example 7

### Verification that Sperm Sex Specific Antigens have been Identified

Indications that sex specific antigens have been identified by “hits” in the various assays are verified as follows. This verification involves two aspects: first that the anticipated molecule is being recognised; and second that the protein recognised is actually on the surface of the cells and also that the protein segregates with sperm bearing the X or Y chromosome. Some of the assays above indicate strongly the characteristics required, for example immunopurification with intact sperm indicates that the molecule is surface exposed. However, the technique does not indicate segregation with the X or Y chromosome. This feature may be established by flow cytometry, PCR and/or FISH analysis as described below. When using flow cytometry, the sperm size distribution is examined as used by Johnson et al. (Johnson, Anim. Reprod. Sci. 60-61:93-107 (2000)). For analysis by PCR, primers specific for the X and Y chromosomes are used with real time PCR to quantitate the distribution of the sex chromosomes with sperm cells (Alves et al., Theriogenology 59:1415-1419 (2003); Kageyama et al., J. Vet. Med. Sci. 66:509-514 (2004); Parati et al., Theriogenology 66:2202-2209 (2006)). Other techniques, such as western blotting and SISCAPA indicate the identity of the molecule being bound by the agent.

### a) Flow Cytometry

In this experimental design, sperm are stained with candidate antibodies that have been shown to bind sperm and the primary antibodies are recognized with Alexa Fluor™ conjugated secondary antibodies (Invitrogen). The cells are simultaneously stained with Hoechst 33342-dye (the dye used for flow cytometric sex sorting of sperm based on DNA content). This approach allows the sperm to be stained for both DNA content and binding agent recognition (Johnson et al. Hum. Reprod. 8:1733-1739 (1993)). Sperm labelled with the candidate antibodies that specifically bind X-chromosome specific antigens will be enriched for sperm that bear the X-chromosome (i.e. those that bind more of the Hoechst dye).

b) Flow Cytometry Sorting Coupled with Real Time PCR

In this study, sperm that bind candidate antibodies on the flow cytometer are sorted into two populations, namely those with staining and those without. DNA is prepared from the two populations and the quantity of X- and Y-chromosome in each sample is determined by real-time PCR for example by the use of the Quantifiler® Duo DNA Quantification Kit (Applied Biosytems) This approach enables accurate relative quantification of X and Y chromosomes present in the two populations.

A variant of this approach is to employ the candidate binding antibodies with magnetic beads to sort the sperm into two populations (binding and non-binding) and then use the flow cytometer to measure DNA content (and hence determine X:Y ratio) or Real-time PCR to indicate the ratio of X- and Y-chromosome on the selected cells.

### c) FISH (Fluorescent In Situ Hybridization) Analysis

A method that allows determination of the X:Y ratio in candidate antibody-bound sperm is to use FISH probes specific for the X- and Y-chromosome. In this approach, sperm are first bound to the primary candidate antibody followed by a fluorescently labelled secondary antibody. The cells are subsequently fixed, permeabilized, and stained with the FISH probes. After a washing step, the cells are viewed under a fluorescent microscope and the X:Y staining ratio of sperm positive for the candidate antibody are determined.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, method, method step or steps, for use in practicing the present invention. All such modifications are intended to be within the scope of the claims appended hereto.

All of the publications, patent applications and patents cited in this application are herein incorporated by reference in their entirety to the same extent as if each individual publication, patent application or patent was specifically and individually indicated to be incorporated by reference in its entirety.

SEQ ID NO: 1-202 are set out in the attached Sequence Listing. The codes for nucleotide sequences used in the attached Sequence Listing, including the symbol “n,” conform to WIPO Standard ST.25 (1998), Appendix 2, Table 1.

I claim:

1. A method for separating of X- and Y-chromosome bearing sperm in a sperm sample, comprising:
(a) contacting the sperm sample with at least one binding agent that specifically binds to an X- or Y-chromosome specific antigen for a period of time sufficient to form a conjugate between the binding agent and the X- or Y-chromosome bearing sperm; and
(b) separating the antibody-sperm conjugate from unbound sperm, wherein the X- or Y-chromosome specific antigen comprises a sequence selected from the group consisting of: (i) SEQ ID NO: 1-21, 43-89, 138, 140, 142, 144, 146, 148, 150, 152, 154, 156, 158, 160, 162, 164 and 183-201; (ii) sequences having at least 85%, 90% or 95% identity to a sequence of SEQ ID NO: 1-21, 43-89, 138, 140, 142, 144, 146, 148, 150, 152, 154, 156, 158, 160, 162, 164 and 183-201; and (iii) sequences encoded by a polynucleotide sequence that hybridizes to a sequence of SEQ ID NO: 22-42, 90-136, 137, 139, 141, 143, 145, 147, 149, 151, 153, 155, 157, 159, 161, 163, 165-182 or 202 under stringent hybridization conditions.

2. The method of claim 1, wherein the binding agent is an antibody or an antigen-binding fragment thereof.

3. The method of claim 2, wherein the binding agent is a Fab or an scFv.

4. The method of claim 2, wherein the antibody is selected from the group consisting of: antibodies identified in Table 1 above.

5. The method of claim 1, wherein the binding agent is attached to a solid support.

6. The method of claim 1, wherein the binding agent is provided on the surface of one or more magnetic beads, and the antibody-sperm conjugate is separated from unbound sperm by application of a magnetic field.

7. The method of claim 1, wherein step (b) comprises contacting the sperm sample with a second binding agent that specifically binds the binding agent that specifically binds to an X- or Y-chromosome specific antigen, the second binding agent being immobilized on a substrate.

8. The method of claim 1, wherein the binding agent is provided with a protease recognition site and the method further comprises contacting the antibody-sperm conjugate with protease after separation from unbound sperm, whereby separated sperm is released from the antibody-sperm conjugate.

9. The method of claim 1, wherein the sperm sample is enriched for X-chromosome bearing sperm.

10. A kit for the separation of X- and Y-chromosome bearing sperm in a sperm sample, comprising: (a) a container holding at least one binding agent specific for an X- or Y-chromosome specific antigen, wherein the X- or Y-chromosome specific antigen comprises a sequence selected from the group consisting of: (i) SEQ ID NO: 1-21, 43-89, 138, 140, 142, 144, 146, 148, 150, 152, 154, 156, 158, 160, 162, 164 and 183-201; (ii) sequences having at least 85%, 90% or 95% identity to a sequence of SEQ ID NO: 1-21, 43-89, 138, 140, 142, 144, 146, 148, 150, 152, 154, 156, 158, 160, 162, 164 and 183-201; and (iii) sequences that are encoded by a polynucleotide sequence that hybridizes to a sequence of SEQ ID NO: 22-42, 90-136, 137, 139, 141, 143, 145, 147, 149, 151, 153, 155, 157, 159, 161, 163, 165-182 or 202 under stringent hybridization conditions; and
(b) instructions for using the kit.

11. The kit of claim 10, wherein the binding agent is an antibody or an antigen binding fragment thereof.

12. The kit of claim 11, wherein the binding agent is a Fab or an scFv.

13. The kit of claim 11, wherein the antibody is selected from the group consisting of:
antibodies identified in Table 1 above.

14. The kit of claim 10 wherein the binding agent is attached to a solid support.

15. The kit of claim 10, wherein the binding agent is provided on the surface of one or more magnetic beads.

16. A composition comprising separated sperm prepared according to the method of claim 1.

17. A method for enriching a semen sample for either X- or Y-chromosome bearing sperm, comprising contacting the semen sample with at least one binding agent that specifically binds to an X- or Y-chromosome specific antigen for a period of time sufficient to form a conjugate between the binding agent and the X- or Y-chromosome bearing sperm, wherein binding of the X- or Y-chromosome bearing sperm to the binding agent is effective in reducing at least one of mobility and activity of the sperm, and wherein the X- or Y-chromosome specific antigen comprises a sequence selected from the group consisting of: (i) SEQ ID NO: 1-21, 43-89, 138, 140, 142, 144, 146, 148, 150, 152, 154, 156, 158, 160, 162, 164 and 183-201; (ii) sequences having at least 85%, 90% or 95% identity to a sequence of SEQ ID NO: 1-21, 43-89, 138, 140, 142, 144, 146, 148, 150, 152, 154, 156, 158, 160, 162, 164 and 183-201; and (iii) sequences encoded by a polynucleotide sequence that hybridizes to a sequence of SEQ ID NO: 22-42, 90-136, 137, 139, 141, 143, 145, 147, 149, 151, 153, 155, 157, 159, 161, 163, 165-182 or 202 under stringent hybridization conditions.

18. The method of claim 17, wherein the binding agent is attached to a cytotoxin.
